(19) United States
(12) Patent Application Publication
(10) Pub. No.: US 2020/0222649 A1
(43) Pub. Date: Jul. 16, 2020

(54) TRACHEOTOMY DEVICE

(71) Applicant: St. Joseph Health System, Orange, CA (US)

(72) Inventors: Byron Wilson, Pasadena, CA (US); Harrison Yen, Irvine, CA (US); Ethan Young, Los Angeles, CA (US); Jeff Eldredge, Los Angeles, CA (US); Tim Colonius, Sierra Madre, CA (US)

(21) Appl. No.: 16/641,211

(22) PCT Filed: Aug. 23, 2018
(86) PCT No.: PCT/US18/47719

§ 371 Date: Feb. 21, 2020

Related U.S. Application Data
(60) Provisional application No. 62/549,663, filed on Aug. 24, 2017.

(51) Int. Cl. A61M 16/04

(52) U.S. Cl. CPC A61M 16/045; A61M 2205/7545; A61M 16/047

(57) ABSTRACT

An aspect of the disclosure is related to a medical device for providing an air passageway through a neck of a patient. In one embodiment, the medical device may include: a neckband configured to extend around the neck of the patient, the neckband including a connector positioned along a length of the neckband for positioning along an anterior part of the neck of the patient, the connector including a connector through hole; a cannula having an elongated tube extending from a connecting head, the elongated tube being configured to extend into a trachea of the patient and including an inner passageway, the connecting head having a through hole that is continuous with the inner passageway of the elongated tube thereby allowing air to pass through the cannula into the trachea, the connecting head having an outer diameter that is sized to have a friction fit with the connector through hole.

### CROSS REFERENCE TO RELATED APPLICATION

The current application claims priority to U.S. Provisional Application No. 62/549,663 filed on Aug. 24, 2017, the contents of which are hereby fully incorporated by reference.

### BACKGROUND

A tracheotomy is a surgical procedure that includes making an incision on the anterior aspect of the neck and opening a direct airway through the incision into the trachea (windpipe). The resulting stoma (hole), or tracheostomy, can serve as an airway or as a site for a tracheal tube to be inserted. A tracheal tube is a catheter that can be inserted into the trachea for the purpose of establishing and maintaining an airway and to ensure the adequate exchange of oxygen and carbon dioxide. For example, a tracheal tube can include a curved metal or plastic tube that is inserted into the stoma to maintain a patent lumen. As such, the tracheal tube can allow a person to breathe without the use of the nose or mouth. Some currently available tracheal devices include a tracheal tube with a proximal end that protrudes out of the stoma for coupling to a restraining element, such as a neck strap, to hold the tracheal tube in place. Such currently available tracheal devices are generally a one-size-fits-all and can be uncomfortable and interfere with at least head movement. Other shortcomings can include cumbersome and large accessories for providing additional features such as air filtering, as well as insufficient or non-ideal air flow. As such, there is a need for improved tracheal tubes and related accessories.

### SUMMARY

A first aspect of the present disclosure is related to a medical device for providing an air passageway through a neck of a patient. The medical device may include: a neckband configured to extend around the neck of the patient, the neckband including a connector positioned along a length of the neckband for positioning along an anterior part of the neck of the patient, the connector including a connector through hole; a cannula having an elongated tube extending from a connecting head, the elongated tube being configured to extend into a trachea of the patient and including an inner passageway, the connecting head having a through hole that is continuous with the inner passageway of the elongated tube thereby allowing air to pass through the cannula into the trachea, the connecting head having an outer diameter that is sized to have a friction fit with the connector through hole.

The neckband can be adjustable in length and can include at least one angled section along a length of the neckband. The at least one angled section may be configured to allow a second section of the neckband to extend between the C2 and C3 vertebrae of the patient when the connector is positioned along the anterior part of the neck. Further, the at least one angled section may be configured to extend over a trapezius muscle of the patient when the connector is positioned along the anterior part of the neck.

The connector may include a front surface that is approximately continuous with an outer surface of the neckband. The outer surface may oppose an inner surface of the neckband, and the inner surface may be configured for positioning against the neck during use of the medical device. In some embodiments, the connector may include an extruded perimeter along a front surface of the connector. The front surface and the extruded perimeter may form an open container. In such an embodiment, a faceplate may releasably couple to an open end of the open container. The faceplate may include a filtered passageway that prevents air particulates from entering the cannula.

In some embodiments, the connecting head may have a substantially circular shaped outer diameter that forms a friction fit with a substantially circular shaped inner diameter of the connector. In another embodiment, the connecting head may have a substantially triangular shaped outer diameter that forms a friction fit with a substantially triangular shaped inner diameter of the connector. In yet another embodiment, the connecting head may have a substantially oval shaped outer diameter that forms a friction fit with a substantially oval shaped inner diameter of the connector.

The through hole may include about a twenty-degree taper extending distally such that an inner diameter of the through hole is largest at a proximal end of the through hole. In some embodiments, at least one vent may be positioned along an outer wall of the connecting head. The at least one vent may allow airflow to pass through the outer wall and into the through hole.

In further embodiments, the medical device may include a stoma guard positioned about the elongated tube and distal to the connecting head. In another embodiment, the medical device may include a stoma guard integrally formed as part of the cannula about the elongated tube and distal to the connecting head. The medical device can also include a cuff and a pilot balloon. The cuff may be positioned about an end of the elongated tube opposing connecting head. The cuff may be configured to be inflated and deflated by the pilot balloon. In an even further embodiment, the medical device can include a filter coupled to the connecting head. The filter may be configured to prevent air particulates from entering the inner passageway of the elongate tube.

A second aspect of the disclosure is related to a medical device for providing an air passageway through a neck of a patient. The medical device may include: an elongated tube having an inner passageway and configured to extend into a trachea of the patient; a connecting head coupled to a proximal end of the elongated tube, the connecting head having a through hole that is continuous with the inner passageway of the elongated tube, the connecting head having a flat shape with a front surface opposed to a back surface, the back surface having an adhesive configured for securing the back surface of the connecting head to an anterior part of the neck. The front surface of the connecting head may include a coupling feature that releasably connects to a filter configured to prevent air particulates from entering the inner passageway of the elongate tube.

### BRIEF DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 shows a perspective view of a trach device according to an embodiment of the disclosure;

FIG. 2 shows a cross-sectional view of the cannula of FIG. 1;

FIG. 3 shows a perspective view of a cannula for a trach device according to an embodiment of the disclosure;

FIG. 4 shows a perspective view of an assembled trach device according to an embodiment of the disclosure including the cannula of FIG. 3;

FIG. 5 shows a perspective view of a trach device according to an embodiment of the disclosure;

FIG. 6 shows a perspective view of a cannula for a trach device according to an embodiment of the disclosure;

FIG. 7 shows a perspective view of an assembled trach device according to an embodiment of the disclosure including the cannula of FIG. 6;

FIG. 8 shows a perspective view of a trach device according to another embodiment of the disclosure;

FIG. 9 shows a perspective view of a trach device according to another embodiment of the disclosure; and

FIG. 10 shows a perspective view of an embodiment of a cannula including a cuff, a pilot balloon, and an inflation tube.

It is noted that the drawings of the subject matter are not necessarily to scale. The drawings are intended to depict only typical aspects of the subject matter, and therefore, should not be considered as limiting the scope of the disclosed subject matter. In the drawings, like numbering represents like elements between the drawings.

### DETAILED DESCRIPTION

The present disclosure describes various embodiments of a tracheotomy device or trach device that includes one or more various improvements over currently available trach devices. For example, some of the embodiments disclosed herein are configured for specific age and/or sized patients, such as a trach device configured for a baby (e.g., up to age 3), a child (e.g., age 3-10 years old), a young adult or teenager (e.g., 10-18 years old), and an adult (e.g., older than 18 years). Some embodiments of the trach device include lower profile connecting ends that protrude from the stoma, thereby improving comfort and reducing or eliminating interference with head movement. Some embodiments of the trach device include improved air flow, improved air filtering, and/or improved restraints for holding the trach device in place. Furthermore, one or more parts the trach devices described herein can be used with one or more parts of currently available trach devices thus allowing adaptability to some currently used trach devices.

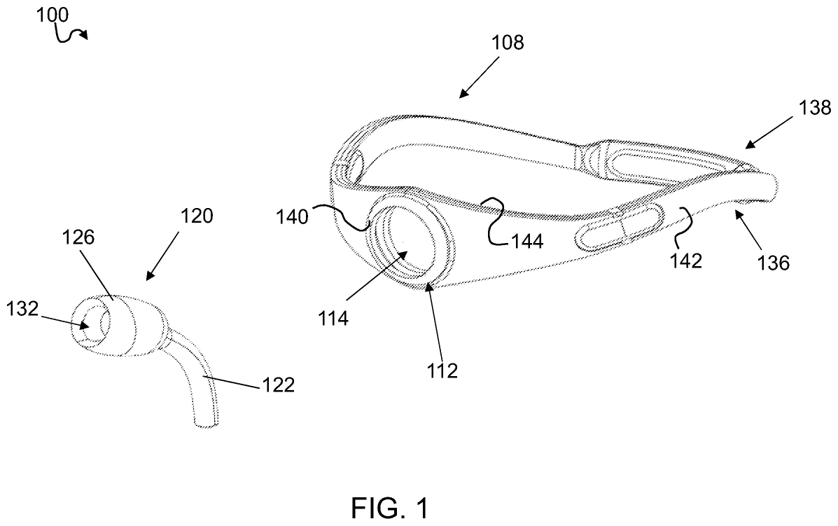

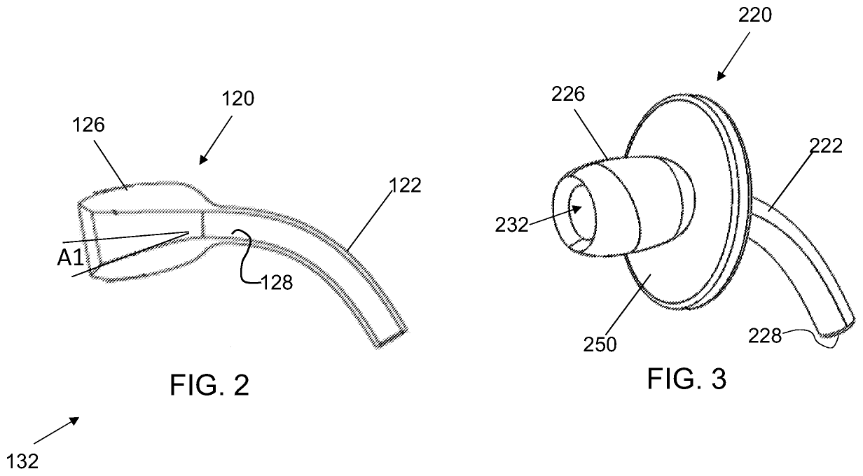

Turning to FIG. 1, a trach device 100 is shown for providing an air passageway through a neck of a patient and includes a neckband 108 configured to extend around the neck of the patient. Neckband 108 can include a connector 112 positioned along a length of neckband 108 for positioning along an anterior part of the neck of the patient. Neckband 108 may be composed of a flexible material, rigid material, a semi-rigid material, or combinations thereof. Neckband 108 may be composed of, e.g., plastic, silicone, metals, composites and fabrics. In some embodiments, neckband 108 may include a dual overmold construction that allows for a soft, comfortable interface while maintaining a desired shape of neckband 108. Connector 112 can include a connector through hole 114 for coupling a cannula 120 thereto. In some embodiments, trach device 100 can further include cannula 120 having an elongated tube 122 extending from a connecting head 126. Elongated tube 122 can be configured to extend into a trachea of the patient and include an inner passageway 128 (FIG. 2). Connecting head 126 can include a through hole 132 that is continuous with inner passageway 128 of the elongated tube 122 thereby allowing air to pass through cannula 120 into the trachea of the patient. Connecting head 126 can have an outer diameter that is sized to have a friction fit with connector through hole 114. This allows for easy placement of cannula 120 and neckband 108 while maintaining a secure fit. In some embodiments, connecting head 126 can have a substantially circular shaped outer diameter that forms a friction fit with a substantially circular shaped inner diameter of connector 112. In other embodiments, connecting head 126 can have a substantially triangular shaped outer diameter that forms a friction fit with a substantially triangular shaped inner diameter of connector 112. In yet another embodiment, connecting head 126 can have a substantially oval shaped outer diameter that forms a friction fit with a substantially oval shaped inner diameter of connector 112. As used herein, the terms “about,” “substantially,” “approximately,” and variations thereof are intended to include the degree of error associated with measurement of the particular quantity based upon the equipment available at the time of filing the application.

The outer diameter of connecting head 126 can also be sized and shaped to fit with standard respiratory connectors, e.g., a ventilator (not shown), where trach device 100 is used as a single cannula trach device. Alternatively, where trach device 100 is used as a double cannula trach device, connecting head 126 and elongated tube 122 may be sized and shaped to accommodate a conventional inner cannula. Through hole 132 can include about a twenty-degree taper A1 extending distally such that an inner diameter of through hole 132 is largest at a proximal end of through hole 132.

In some variations one or more of the following features can optionally be included in any feasible combination. For example, neckband 108 can be adjustable in length. In some embodiments, neckband 108 may include plastic fastener straps, similar to those found on snapback hats. In other embodiments, neckband 108 can include a tie, Velcro, or clasp for adjusting a length. Neckband 108 can also include at least one angled section 136 along a length of neckband 108, and at least one angled section 136 can be configured to allow a rear section 138 of neckband 108 to extend between the C2 and C3 vertebrae of the patient when connector 112 is positioned along the anterior part of the patient's neck. In some implementations, the at least one angled section 136 can be configured to extend over a trapezius muscle of the patient when connector 112 is positioned along the anterior part of the neck.

In some embodiments, connector 112 can include a front surface 140 that is approximately continuous with an outer surface 142 of neckband 108, and outer surface 142 can oppose an inner surface 144 of neckband 108. Inner surface 144 of neckband 108 can be configured for positioning against the neck of the patient during use of trach device 100.

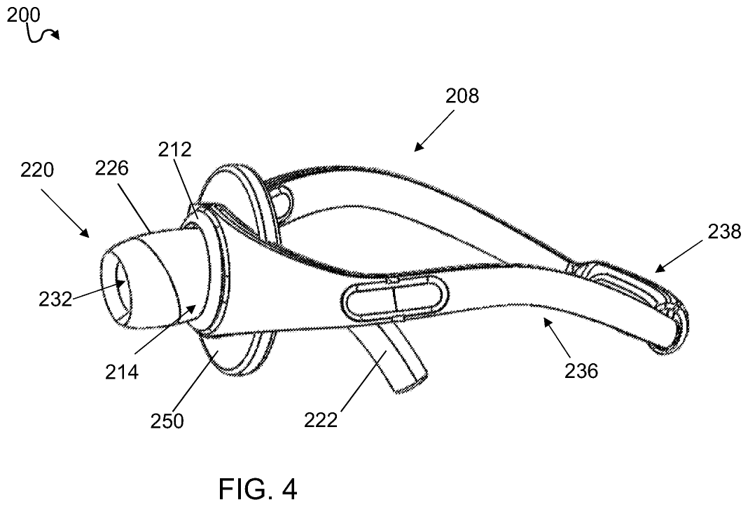

FIGS. 3-4 show a cannula 220 according to another embodiment of the disclosure. In this embodiment, cannula 220 may include having an elongated tube 222 extending from a connecting head 226. Elongated tube 222 can be configured to extend into a trachea of the patient and include an inner passageway 228 similar to inner passageway 128 shown in FIG. 2. Connecting head 226 can include a through hole 232 that is continuous with the inner passageway 228 of the elongated tube 222 thereby allowing air to pass through cannula 220 into the trachea of the patient. Cannula 220 of this embodiment may further include a flange or stoma guard 234. Stoma guard 250 can be connected, affixed, and/or attached to connecting head 226 and/or elongated tube 222. For example, stoma guard 250 may be positioned about elongated tube 222 and distal to connecting head 226. In other embodiments, stoma guard 250 may be integrally formed as part of the cannula 220 about elongated tube 222 and distal to connecting head 226. Stoma guard 250 may prevent connecting head 226 from entering and expanding the stoma of the patient when cannula 220 is in use.

FIG. 4. shows an assembled trach device 200 including cannula 220 of FIG. 3. Like trach device 100 (FIG. 1), trach device 200 includes a neckband 208 having a connector 212 positioned along a length of neckband 208 for positioning along an anterior part of the neck of the patient. Connector 212 can include a connector through hole 214 for coupling cannula 220 thereto. Connecting head 226 of cannula 220 can have an outer diameter that is sized and shaped to have a friction fit with connector through hole 214. This allows for easy placement of cannula 220 and neckband 208 while maintaining a secure fit. Neckband 208 can be adjustable in length. In some embodiments, neckband 208 may include plastic fastener straps, similar to those found on snapback hats. In other embodiments, neckband 208 can include that tie, Velcro, or clasp together. Neckband 208 can also include at least one angled section 236 along a length of neckband 208, and at least one angled section 236 can be configured to allow a rear section 238 of neckband 208 to extend between the C2 and C3 vertebrae of the patient when connector 212 is positioned along the anterior part of the patient's neck. In some implementations, the at least one angled section 236 can be configured to extend over a trapezius muscle of the patient when connector 212 is positioned along the anterior part of the neck.

Trach device 200 according to this embodiment, can be assembled by positioning cannula 220 within a stoma of a patient such that elongated tube 222 extends into a trachea of the patient and stoma guard 234 abuts the neck of the patient at about the stoma. Once cannula 220 is positioned, neckband 208 may be positioned around the neck of the patient such that connecting head 226 extends through connecting hole 214 and extends in a proximal direction relative to neckband 208 away from the patient's neck. Connecting head 226 may form a friction fit with connector 212 within through hole 214. Connecting head 226 can extend proximally through connecting hole 214 enough to allow connecting head 226 to be connected to standard respiratory connectors, if needed.

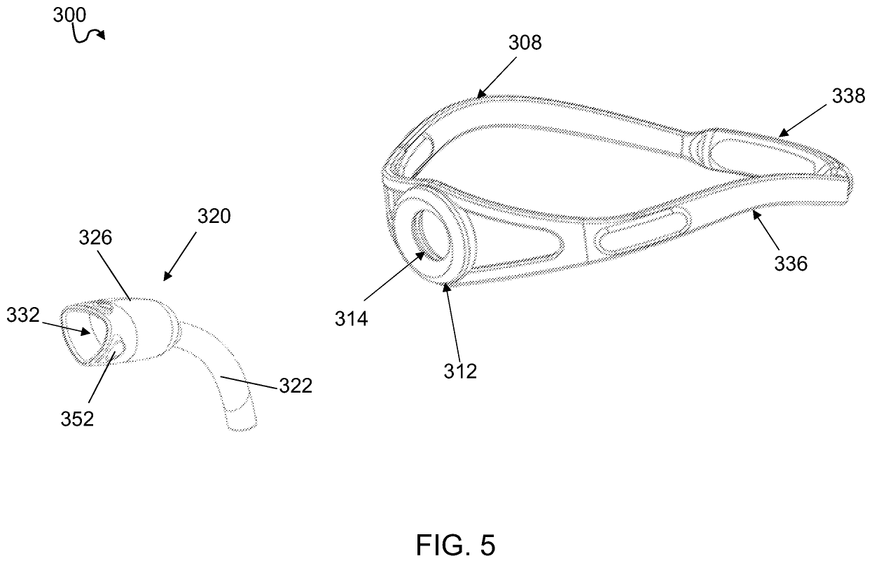

FIG. 5 shows a trach device 300 according to another embodiment of the disclosure. In this embodiment, trach device 300 can include neckband 308 having connector 312, connector through hole 314, angled section 336, and rear portion 338 similar to trach devices 100, 200 (FIGS. 1 and 4). In addition, trach device 300 may include cannula 320 having a substantially triangular shaped connecting head 326 including a through hole 332 therein for providing air to a trachea of a patient through elongated tube 322. However, in this embodiment, connecting head 326 may further have at least one vent 352 formed about an outer perimeter of connecting head 326. Vent 352 provide allow airflow to pass through the outer wall of connecting head 326 thereby providing increased airflow to the trachea of the patient.

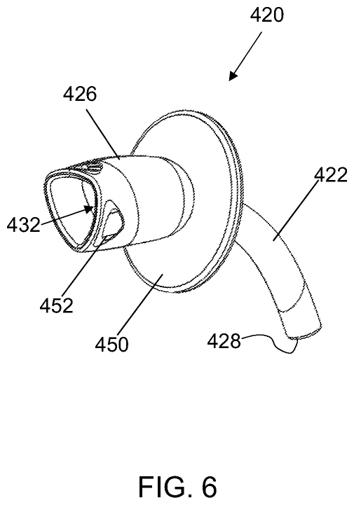

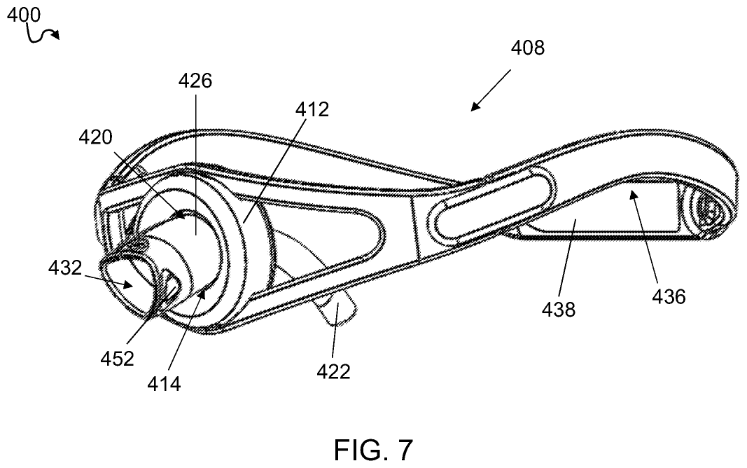

FIGS. 6-7 show a cannula 420 according to another embodiment of the disclosure. In this embodiment, cannula 420 may include having an elongated tube 422 extending from a connecting head 426. Elongated tube 422 can be configured to extend into a trachea of the patient and include an inner passageway 428 similar to inner passageway 128 shown in FIG. 2. Connecting head 426 can include a through hole 432 that is continuous with the inner passageway 428 of the elongated tube 422 thereby allowing air to pass through cannula 420 into the trachea of the patient. Stoma guard 450 can be connected, affixed, and/or attached to connecting head 426 and/or elongated tube 422. For example, stoma guard 450 may be positioned about elongated tube 422 and distal to connecting head 426. In other embodiments, stoma guard 450 may be integrally formed as part of the cannula 420 about elongated tube 422 and distal to connecting head 426. Stoma guard 450 may prevent connecting head from entering and expanding the stoma of the patient when cannula 420 is in use.

FIG. 7. shows an assembled trach device 400 including cannula 420 of FIG. 6. Like trach devices 100, 200, 300 (FIGS. 1 and 4-5), trach device 400 includes a neckband 408 having a connector 412 positioned along a length of neckband 408 for positioning along an anterior part of the neck of the patient. Connector 412 can include a connector through hole 414 for coupling a cannula 420 thereto. Connecting head 426 of cannula 420 can have an outer diameter that is sized to have a friction fit with connector through hole 414. This allows for easy placement of cannula 420 and neckband 408 while maintaining a secure fit. Neckband 408 can be adjustable in length. In some embodiments, neckband 408 may include plastic fastener straps, similar to those found on snapback hats. In other embodiments, neckband 408 can include that tie, Velcro, or clasp together. Neckband 408 can also include at least one angled section 436 along a length of neckband 408, and at least one angled section 436 can be configured to allow a rear section 438 of neckband 408 to extend between the C2 and C3 vertebrae of the patient when connector 412 is positioned along the anterior part of the patient's neck. In some implementations, the at least one angled section 436 can be configured to extend over a trapezius muscle of the patient when connector 412 is positioned along the anterior part of the neck.

Trach device 400 according to this embodiment, can be assembled by first positioning cannula 420 within a stoma of a patient such that elongated tube 422 extends into a trachea of the patient and stoma guard 434 abuts the neck of the patient at about the stoma. Once cannula 420 is positioned, neckband 408 may be positioned around the neck of the patient such that connecting head 426 extends through connecting hole 414 and extends in a proximal direction away from the patient's neck. Connecting head 426 may form a friction fit with connector 412 within connecting hole 414. Connecting head 426 can extend proximally through connecting hole 414 enough to allow connecting head 426 to be connected to a ventilator, if needed.

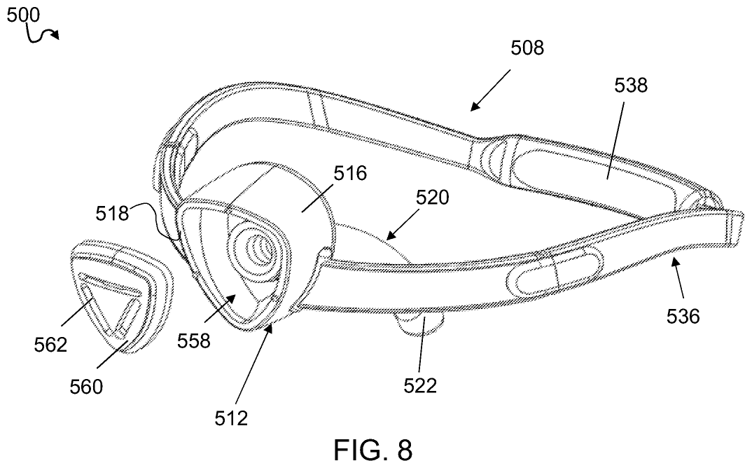

FIG. 8 shows another embodiment of a trach device 500 according to the disclosure. In this embodiment, trach device 500 can include neckband 508 for positioning along an anterior part of the neck of the patient. A connector 512 may be positioned along a length of neckband 508. Connector 512 may be integrally formed with neckband 508 in some embodiments. However, in other embodiments, connector 512 may be releasably coupled to neckband 508 as will be described herein. Cannula 520 together with elongated tube 522 may be integrally formed with connector 512. However, in other embodiments, elongated tube 522 may be removably coupled with connector 512. Connector 512 in this embodiment can include an extruded perimeter 516 along front surface 518 of connector 512 thereby forming an open container 558 together with cannula 520.

Neckband 508 can be releasably coupled to connector 512 on opposing sides of connector 512. This allows removal of neckband 508 from connector 512, and therefore, from the patient while maintaining the position of cannula 520 within the neck of the patient. Neckband 508 can be releasably coupled to connector 512 via, for example, clasps, snap-fit connections, ties, magnets, fasteners, or friction fit.

Trach device 500 can include a faceplate 560 that releasably couples to an open end of extruded perimeter 516 of connector 512, or open container 558. Faceplate 560 can include a filtered passageway 562 that prevents air particulates from entering cannula 520. In addition, cannula 520 may be sized and shaped such that cannula 520 may be used together with a conventional inner cannula (not specifically shown here).

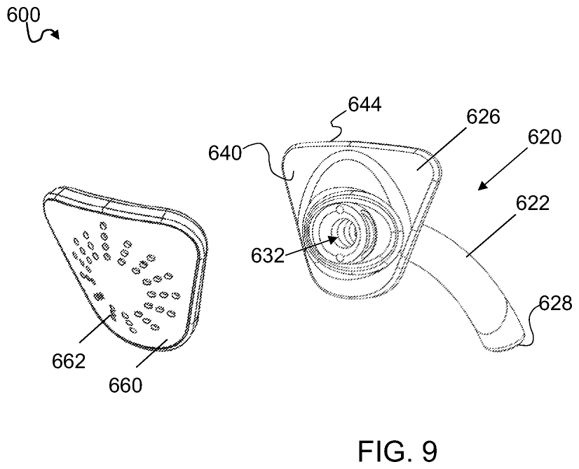

In another aspect of the disclosure, trach device 600 (FIG. 9) can include a cannula 620 having an elongated tube 622 with an inner passageway 628 therein and configured to extend into a trachea of the patient. Trach device 600 can further include a connecting head 626 coupled to a proximal end of elongated tube 622. Connecting head 626 can have a through hole 632 that is continuous with inner passageway 628 of elongated tube 622. In addition, connecting head 626 can have a substantially flat shape with a front surface 640 opposed to a back surface 644, and back surface 644 can have an adhesive configured for securing back surface 644 of connecting head 626 to an anterior part of the neck. In this way, trach device 600 can include an all-in-one stoma guard (i.e., connecting head 626) and cannula 620 that can be used with an adhesive/second skin attachment to the neck. For example, trach device 600 can be used without a neck strap to thereby provide a very low-profile and discreet device for wearing by a patient. In some embodiments, front surface 640 of connecting head 626 can include a coupling feature that releasably connects to a faceplate 660 having a filtered passageway 662 configured to prevent air particulates from entering inner passageway 628 of elongate tube 628. However, in other embodiments, faceplate 660 may include a raised edge for coupling with and/or matingly engaging with an outer perimeter of connecting head 626. Faceplate 660 can be coupled to connecting head 626 via, e.g., snap fit, friction fit, and/or magnets. In other embodiments, faceplate 660 can be composed at least partially of silicone so that faceplate 660 stretches over connecting head 626. In addition, a gasket (not individually shown) can be provided at an interface of faceplate 660 and connecting head 626 in some embodiments.

Furthermore, cannula 620 of trach device 600 can include an outer cannula that can be sized and shaped to fit with a conventional inner cannula (not individually shown). For example, cannula 620 can connect and/or extend from connecting head 626 (i.e., the stoma guard in this embodiment), which can have various shapes and sizes. Additionally, an inner cannula can fit at least partly inside of cannula 620 to thereby be held in place, such as by either friction of some mechanical fit.

Any of the trach devices disclosed herein can be specifically sized and shaped to suit a particular age and/or size of the patient. For example, FIGS. 1-4 may be configured for use by a baby (e.g., up to age 3. FIGS. 5-7 may be configured for use by a child. (e.g., age 3-10 years old). FIG. 8 may be configured for use by a young adult or teenager (e.g., 10-18 years old). FIG. 9 may be configured for use by an adult (e.g., older than 18 years). Although the embodiments of the trach device described and shown in the figures may be identified as being configured for a type of patient (e.g., an age group), all of the trach embodiments described and shown herein, including any one or more features of a single embodiment, can be included in a trach device embodiment configured for any type of patient (e.g., any age group) without departing from the scope of this disclosure.

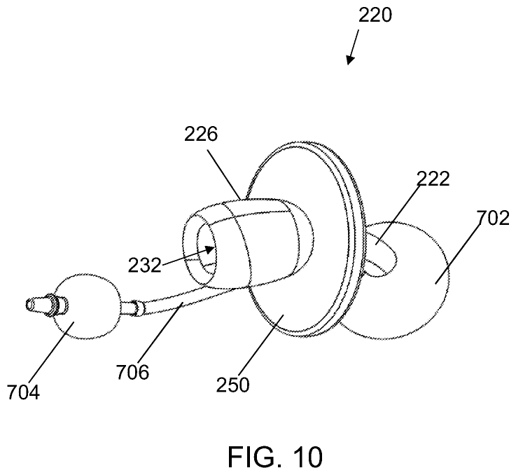

In addition, other features can be incorporated into any of the trach devices disclosed herein. For example, FIG. 10 shows cannula 220 of FIGS. 3-4 including the additional features of a cuff 702, a pilot balloon 704, and an inflation tube 706. Cuff 702 may include a small inflatable balloon that is configured to be inflated and deflated by pilot balloon 704. Cuff 702 can be filled with a fluid (e.g., air of sterile water) to inflate cuff 702. Cuff 702 may be positioned about an end of elongated tube 222 opposing connecting head 226. Cuff 702 may assist in sealing of the patient's trachea when ventilation is needed, and can also help support the position of elongated tube 22 within the patient's trachea. Pilot balloon 704 can be used for expanding cuff 704 and inflates in relation to cuff 702. An inflation tube 706 may be fluidly coupled to each of cuff 702 and pilot balloon 704. Pilot balloon 704 can include a small inflatable bladder and can be positioned exterior to the patient's neck about an end of inflation tube 706 that is opposite of cuff 702. During use, a syringe (not specifically shown) may be used to inflate and deflate pilot balloon 704 thereby inflating and deflating cuff 702.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The implementations set forth in the foregoing description do not represent all implementations consistent with the subject matter described herein. Instead, they are merely some examples consistent with aspects related to the described subject matter. Although a few variations have been described in detail herein, other modifications or additions are possible. In particular, further features and/or variations can be provided in addition to those set forth herein. For example, the implementations described above can be directed to various combinations and sub-combinations of the disclosed features and/or combinations and sub-combinations of one or more features further to those disclosed herein. In addition, the logic flows depicted in the accompanying figures and/or described herein do not necessarily require the particular order shown, or sequential order, to achieve desirable results. The scope of the following claims may include other implementations or embodiments.

What is claimed is:

1. A medical device for providing an air passageway through a neck of a patient, the medical device, comprising:
a neckband configured to extend around the neck of the patient, the neckband including a connector positioned along a length of the neckband for positioning along an anterior part of the neck of the patient, the connector including a connector through hole;
a cannula having an elongated tube extending from a connecting head, the elongated tube being configured to extend into a trachea of the patient and including an inner passageway, the connecting head having a through hole that is continuous with the inner passageway of the elongated tube thereby allowing air to pass through the cannula into the trachea, the connecting head having an outer diameter that is sized to have a friction fit with the connector through hole.

2. The medical device of claim 1, wherein the neckband is adjustable in length.

3. The medical device of claim 1, wherein the neckband includes at least one angled section along a length of the neckband.

4. The medical device of claim 3, wherein the at least one angled section is configured to allow a second section of the neckband to extend between the C2 and C3 vertebrae of the patient when the connector is positioned along the anterior part of the neck.

5. The medical device of claim 3, wherein the at least one angled section is configured to extend over a trapezius muscle of the patient when the connector is positioned along the anterior part of the neck.

6. The medical device of claim 1, wherein the connector includes a front surface that is approximately continuous with an outer surface of the neckband, wherein the outer surface opposes an inner surface of the neckband, and wherein the inner surface is configured for positioning against the neck during use of the medical device.

7. The medical device of claim 1, wherein the connector includes an extruded perimeter along a front surface of the connector, wherein the front surface and the extruded perimeter form an open container.

8. The medical device of claim 7, further comprising: a faceplate that releasably couples to an open end of the open container, wherein the faceplate includes a filtered passageway that prevents air particulates from entering the cannula.

9. The medical device of claim 1, wherein the connecting head has a substantially circular shaped outer diameter that forms a friction fit with a substantially circular shaped inner diameter of the connector.

10. The medical device of claim 1, wherein the connecting head has a substantially triangular shaped outer diameter that forms a friction fit with a substantially triangular shaped inner diameter of the connector.

11. The medical device of claim 1, wherein the connecting head has a substantially oval shaped outer diameter that forms a friction fit with a substantially oval shaped inner diameter of the connector.

12. The medical device of claim 1, wherein the through hole includes about a twenty-degree taper extending distally such that an inner diameter of the through hole is largest at a proximal end of the through hole.

13. The medical device of claim 1, further comprising: at least one vent along an outer wall of the connecting head, wherein the at least one vent allows airflow to pass through the outer wall and into the through hole.

14. The medical device of claim 1, further comprising: a stoma guard positioned about the elongated tube and distal to the connecting head.

15. The medical device of claim 1, further comprising: a stoma guard integrally formed as part of the cannula about the elongated tube and distal to the connecting head.

16. The medical device of claim 1, further comprising: a cuff and a pilot balloon, wherein the cuff is positioned about an end of the elongated tube opposing connecting head, and wherein the cuff is configured to be inflated and deflated by the pilot balloon.

17. The medical device of claim 1, further comprising: a filter coupled to the connecting head, the filter configured to prevent air particulates from entering the inner passageway of the elongate tube.

18. A medical device for providing an air passageway through a neck of a patient, the medical device, comprising:
an elongated tube having an inner passageway and configured to extend into a trachea of the patient;
a connecting head coupled to a proximal end of the elongated tube, the connecting head having a through hole that is continuous with the inner passageway of the elongated tube, the connecting head having a flat shape with a front surface opposed to a back surface, the back surface having an adhesive configured for securing the back surface of the connecting head to an anterior part of the neck.

19. The medical device of claim 18, wherein the front surface of the connecting head includes a coupling feature that releasably connects to a filter configured to prevent air particulates from entering the inner passageway of the elongate tube.
